(19) United States
(12) Patent Application Publication
(10) Pub. No.: US 2023/0323008 A1
(43) Pub. Date: Oct. 12, 2023

(54) SPECIAL IMINES AND THEIR STARTING MATERIALS, AND THEIR USE IN THE HARDENING OF REACTIVE RESINS BY POLYADDITION OR FREE-RADICAL POLYMERISATION

(71) Applicant: fischerwerke GmbH & Co. KG, Waldachtal (DE)

(72) Inventors: Christian WEINELT, Teningen (DE); Martin VOGEL, Waldkirch (DE)

(21) Appl. No.: 18/012,950

(22) PCT Filed: Jun. 14, 2021
(86) PCT No.: PCT/EP2021/065917

§ 371 Date: Dec. 26, 2022

(30) Foreign Application Priority Data
Jul. 2, 2020 (DE) 10 2020 117 416.0
Jun. 9, 2021 (DE) 10 2021 114 890.1

(51) Int. Cl. C08G 18/32; C08G 18/10; C07C 251/08; C09D 175/02; C09J 175/02

(52) U.S. Cl. CPC C08G 18/3256; C07C 251/08; C08G 18/10; C09D 175/02; C09J 175/02

(57) ABSTRACT

Imine, obtainable by reaction of (i) an amino-functionalised polyoxyalkylene, the polyoxyalkylene chains of which are a copolymer of oxyethylene and oxypropylene units, those polyoxyalkylene chains carrying primary amino groups (preferably two terminal primary amino groups per molecule), and (ii) a ketone and/or aldehyde having a hydrogen atom at the carbon atom in the alpha-position to the carbonyl carbon; and various subjects of the invention based thereon, especially in coating systems or adhesive systems.

The invention relates to a special type of imines which is suitable as part of a hardening system for free-radical-polymerisable reactive resins and/or for reactive resins that are hardenable by polyaddition in coating systems and/or adhesive systems.

A number of adhesives and coating materials are available on the market which are based, on the one hand, on free-radical-hardenable compounds or, on the other hand, on compounds that are hardenable by polyaddition (such as, for example, monomers, oligomers or prepolymers carrying two or more isocyanate groups and/or epoxy groups (per molecule)). The polymerisation (curing) of the said adhesives or coating materials is in each case effected by means of hardener systems which, in the former case, comprise free-radical initiators and, in the latter case, comprise amines carrying two or more amino groups and optionally further constituents.

Imines and their precursors (amines and ketones or aldehydes) as constituents of such hardener systems for free-radical-hardenable adhesives are known from WO 2016/206777 A.

The problem was to provide further synthetic resin systems suitable for versatile use in the field of coatings and fixing technology which (in the case of the fixing of anchoring means in drilled holes) give rise to improved properties - especially increased reactivity in comparison with previously known and comparable systems and, above all, improved bond stresses. A further problem was to provide an imine which, in respect of its hazard labelling, is superior to the imines commercially available hitherto (that is to say it does not require hazard labelling) and with which very good tensile shear strengths are nevertheless obtained in the case of adhesive bonds, such as, for example, beech-to-beech bonds.

It has surprisingly been found that certain imines, namely those which are obtainable by reaction of
an amino-functionalised polyoxyalkylene, the polyoxyalkylene chains of which are a copolymer of oxyethylene and oxypropylene units, those polyoxyalkylene chains carrying (preferably terminal) primary amino groups, with
a ketone and/or preferably an aldehyde having a hydrogen atom at the carbon atom in the alpha-position to the carbonyl group (—C(═O)—,
provide an improvement in those properties in comparison with imines known from WO 2016/206777 A1. Moreover, such imines can also be used for the hardening of, for example, epoxides or isocyanates for coatings or adhesive bonds.

In a first embodiment, the invention consequently relates to an imine, obtainable by reaction of (i) an amino-functionalised polyoxyalkylene, the polyoxyalkylene chains of which are a copolymer of oxyethylene and oxypropylene units, those polyoxyalkylene chains carrying primary amino groups (preferably two terminal primary amino groups per molecule), and (ii) a ketone and/or (preferably) aldehyde having a hydrogen atom (preferably a (=1) single hydrogen atom) at the carbon atom in the alpha-position to the carbonyl carbon.

In a second embodiment, the invention relates to an imine as mentioned above, which is obtainable by reaction of the amino-functionalised polyoxyalkylene (i), which carries a primary amino group in each case bonded to the terminal oxyalkylene group, with an alkanaldehyde (ii), especially a C₂-C₈alkanaldehyde, especially one which is singly branched in the alpha-position to the carbonyl group, for example which carries a methyl group in that position. Special preference is given to isobutyraldehyde.

In a third embodiment, the invention relates to an imine as defined above, characterised in that the amino-functionalised polyoxyalkylene (i) carries two 2-aminopropyloxy groups which are terminal in each case.

In a fourth embodiment, the invention relates to an imine as defined above, characterised in that the amino-functionalised polyoxyalkylene (i) has the following formula:

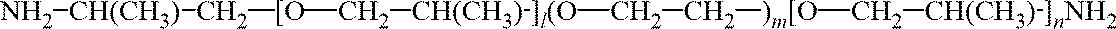

wherein l and n each independently of the other denote, on average, from 1 to 10, preferably from 3 to 4, especially approximately 3.6; and m denotes from 1 to 50, preferably from 8 to 10, especially approximately 9. Preferably, in the formula above, on average, l and n denote approximately 3.6 and, on average, m denotes approximately 9, with “approximately” preferably denoting a range of variation of ± 0.2, especially of ± 0.1.

In a fifth embodiment, the invention relates to an imine as defined in one of the above embodiments, characterised in that it has an average molecular weight of 2000 g/mol or less, for example of 1000 g/mol or less.

A sixth embodiment of the invention relates to an imine as defined in one of the above embodiments in admixture (especially in equilibrium admixture) with its starting materials (i) amino-functional polyoxyalkylene and (ii) ketone and/or (preferably) aldehyde. This variant is an option in the case of free-radical curing (as free-radical initiator).

A seventh embodiment of the invention relates to a mixture of (i) an amino-functional polyoxyalkylene and (ii) an aldehyde and/or ketone, preferably an aldehyde; in each case as defined in one of the above embodiments.

An eighth embodiment of the invention relates to a mixture as defined in the preceding paragraph as constituent of a coating system or adhesive system (or adhesive).

A ninth embodiment of the invention relates to a hardener which
in the case of the free-radical-hardenable compounds (Case A) includes the following initiator system:
   (a) at least one activator in the form of a metal salt and as free-radical starter
   (b1) an amino-functional polyoxyalkylene (i) as defined in one of the above embodiments in admixture with an aldehyde and/or ketone (ii), preferably with an aldehyde, in each case as defined in one of the above embodiments; or
   (b2) an imine as defined in one of the above embodiments,
   or a mixture of constituents (b1) and (b2); or
in the case of the compounds that are hardenable by polyaddition (Case B) represents the following constituent: (b2) an imine as defined in one of the above embodiments.

In the case of the polyaddition, the hardening takes place by means of the amine freed from the imine (in the presence of moisture, (for example atmospheric moisture or moisture in the substrate to be adhesively bonded); that is to say water), which amine is added to an isocyanate and/or epoxy group, preferably isocyanate.

In a tenth embodiment, the invention relates to a coating system or, especially, adhesive system, including a hardener (initiator system), as described for the ninth embodiment, and, as reactive resin, either (Aa) a free-radical-polymerisable reactive resin or (Ba) a reactive resin that is hardenable by polyaddition, or a mixture of (Aa) and (Ba).

In an eleventh embodiment, the invention relates to a coating system or, especially, adhesive system in accordance with the preceding paragraph, wherein the reactive resin is a free-radical-hardenable reactive resin according to (Aa).

A twelfth embodiment of the invention relates to a coating system or, especially, adhesive system in accordance with the penultimate paragraph, wherein the reactive resin is a reactive resin that is hardenable by polyaddition according to (Ba) (as mentioned in the tenth embodiment of the invention), especially an oligomer and/or prepolymer that includes at least two isocyanate groups.

In a thirteenth embodiment, the invention relates to a coating system or, especially, adhesive system in accordance with one of the last three paragraphs in the form of a multi-component system, especially a two-component system, which includes a reactive resin according to (Aa) or (Ba) in one component and a hardener according to the tenth embodiment in another component.

A fourteenth embodiment of the invention relates to a coating system or, especially, adhesive system according to the twelfth embodiment in the form of a single-component system, preferably in the form of a surface-coating that cures by polyaddition, especially in the presence of moisture (water, as such or in aqueous solution with another solvent or solvent mixture), or in the form of an adhesive that cures by polyaddition.

In a fifteenth embodiment, the invention relates to the use of an adhesive system as defined in one of embodiments ten to fourteen
(a) for adhesively bonding articles to one another or
(b) for adhesively bonding an article to a fixed substrate.

A sixteenth embodiment of the invention relates to a use in accordance with the preceding paragraph, variant (b), wherein
an article is to be understood as being an accessory for a swimming pool, bedroom, bathroom or kitchen or an adapter element for holding such an accessory; a plate or a block, preferably made of metal, glass, plastics, concrete or stone; fibres, especially high-modulus fibres, preferably carbon fibres, especially for reinforcing built structures, for example walls, pillars, ceilings or floors; a structural element, such as a panel, a disc, or a block, for example made of stone, glass or plastics; or - in each case implemented in planar form as a mat and as loose material or as composite fabric or material - a non-woven, a woven fabric, a knitted fabric, a planar material obtained by knitting, a material obtained by tufting, preferably in each case with high-modulus fibres, for example made of glass fibres, carbon fibres and/or synthetic fibres, in the case of a composite material having a synthetic matrix; and
a substrate is to be understood as being a building substrate, especially a wall, a ceiling or a floor in the construction field, preferably made of masonry, concrete, unglazed or glazed ceramics, or furthermore made of glass, wood, metal or plastics.

A seventeenth embodiment of the invention relates to a use in accordance with the preceding paragraph of an adhesive system as defined in one of embodiments ten to fourteen for adhesively bonding an anchoring element in a hole or a recess in the substrate, especially in a drilled hole.

An eighteenth embodiment of the invention relates to a use in accordance with the penultimate paragraph of a coating system as defined in one of embodiments ten to fourteen for coating a substrate, the substrate preferably being a building substrate.

In a nineteenth embodiment, the invention relates to a method for coating a substrate with a coating system as defined in one of embodiments ten to fourteen, wherein the coating system is applied, if necessary with or after mixing of components thereof that do not cure on their own, and cured, with polymerisation taking place.

A twentieth embodiment of the invention relates to a method
(a) for adhesively bonding articles to one another or
(b) for adhesively bonding an article to a fixed substrate,
   wherein an adhesive system as defined in one of embodiments ten to fourteen is applied, if necessary with or after mixing of components thereof that otherwise do not react with one another, to one or more areas to be adhesively bonded of one or more of the articles to be adhesively bonded, and areas to be adhesively bonded are brought into contact with one another and the adhesive system is cured, with polymerisation taking place,
the method especially including the use as in one of embodiments fourteen to seventeen.

The invention relates also to the subjects of the invention mentioned in the independent claims and especially in the dependent claims, which are preferably to be regarded herein as incorporated into the description by reference.

The more specific definitions mentioned hereinabove and hereinbelow of individual features or of some of the features or of all of the features of a subject of the invention can be used to replace more specific features, resulting in preferred subjects of the invention (embodiments of the invention) which are likewise subject of the present invention.

Where weights are given in percent (% by weight), unless otherwise indicated or apparent these relate to the total mass of the reactants and additives of all components (liquid or paste-form in the finished formulated state) of the coating system or adhesive system, that is to say without packaging, that is to say the mass (by weight) of the or all associated component constituent(s).

Where the attribute “furthermore” is mentioned, this means that features without this attribute are more preferred.

“And/or” means that the mentioned features/substances can in each case be present on their own or in a combination of two or more of the individually mentioned features/substances.

Where “on average” is mentioned, this relates to the number of the respective groups in relation to the number of molecules that include the group in question, that is to say the arithmetic mean.

“Include” or “comprise” or “contain” means that other components or features can be present in addition to the components or features mentioned and therefore does not refer to an exhaustive list, unlike “consisting of”, the use of which does signify an exhaustive list of the components or features. In special embodiments of the subjects of the invention, the exhaustively defining phrase “consisting of” can be used instead of “include” or “comprise”.

“Obtainable” by a reaction can especially be replaced by “obtained” by the reaction.

Where “a” or “an” is used, this is to be primarily understood (unless otherwise apparent, for example by being preceded by “at least”) as the indefinite article and includes “one (in figures: 1) or more” and also just one (in figures: 1). In other words, “a” or “an” means “one or more, for example two or three or four”. “At least one” denotes one or more. Where the plural is used, this also includes the singular.

The phrase “having a hydrogen atom at the carbon atom in the alpha-position to the carbonyl group” means having at least one hydrogen atom at the carbon atom in the alpha-position to the carbonyl group, preferably having one (1) or two or, furthermore, three hydrogen atom(s), especially just one hydrogen atom, in the said alpha-position (at the carbon vicinal to the carbonyl carbon).

A free-radical-hardenable reactive resin (Aa) is especially one which comprises a compound that includes non-aromatic unsaturated groups (double bonds), preferably a free-radical-hardenable unsaturated reactive resin having preferably at least 2 or more reactive non-aromatic unsaturated bonds, or a mixture of two or more such reactive resins.

The group of the ethylenically unsaturated compounds which comprises styrene and derivatives; vinyl esters, such as (meth)acrylates, urethane (meth)acrylates or itaconates, or epoxy(meth)acrylates; furthermore unsaturated polyesters, vinyl ethers, allyl ethers, dicyclopentadiene compounds and unsaturated fats, is especially suitable.

Particular preference is given primarily to one or more such reactive resins which comprise (free-radically) hardenable esters with one or more unsaturated carboxylic acid radicals (as described, for example, in DE 10 2014 103 923 A1); preferably in each case propoxylated or, especially, ethoxylated aromatic diol-, such as bisphenol-A-, bisphenol-F- or novolak-(especially di-)(meth)acrylates; epoxy(meth)acrylates, especially in the form of reaction products of di- or poly-epoxides, for example bisphenol-A-, bisphenol-F- or novolak-di- and/or -poly-glycidyl ethers, with unsaturated carboxylic acids, for example C₂-C₇alkenecarboxylic acids, such as especially (meth)acrylic acid; urethane- and/or urea-(meth)acrylates - especially urethane (meth)acrylates, which are obtained, for example, by reaction of di- and/or polyisocyanates (higher-functional isocyanates) with suitable (meth)acrylic compounds (such as, for example: hydroxyethyl- or hydroxypropyl-methacrylate), optionally with the participation of hydroxy compounds that contain at least two hydroxy groups, as described, for example, in DE 39 40 309 A1 and/or DE 41 11 828 A1; or unsaturated polyester resins, or the like, or two or more of such hardenable unsaturated organic components.

Examples of epoxy(meth)acrylates used or present in special embodiments of the invention are those of the formula (l)

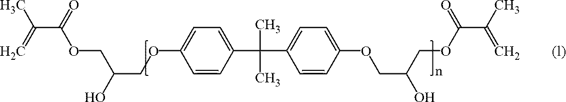

in which n denotes a number greater than or equal to 1 (when mixtures of different molecules having different n values are present and are represented by the formula, non-integer numbers are also possible as a mean value).

Examples of propoxylated or, especially, ethoxylated aromatic diol-, such as bisphenol-A-, bisphenol-F- or novolak-(especially di-)(meth)acrylates that can be used in special embodiments of the invention are those of the formula (ll)

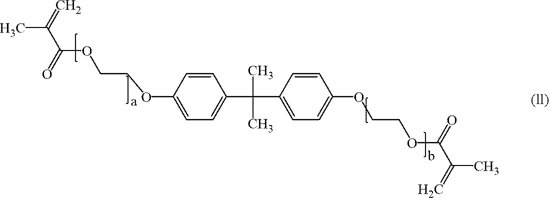

in which a and b each independently of the other denote a number greater than or equal to 0, with the proviso that preferably at least one of the values is greater than 0, preferably both values being 1 or more (when mixtures of different molecules having different (a and b) values are present and are represented by the formula, non-integer numbers are also possible as a mean value), or, in the case of the propoxylated compounds, having branched or unbranched oxypropylene groups instead of O—CH₂—CH₂— in the above formula.

Examples of urethane (meth)acrylates used or present in other special and preferred embodiments of the invention in free-radical-hardenable urethane-methacrylate-based synthetic resins (“vinyl ester urethanes”) in coating systems or adhesive systems according to the invention are those which result, on the one hand, from the reaction of a prelengthened monomeric di- or poly-isocyanate and/or, on the other hand, from the reaction of a polymeric di- or poly-isocyanate (for example: PMDI, MDI) with hydroxyalkyl (meth)acrylate, such as hydroxyethyl or hydroxypropyl (meth)acrylate. The way in which prelengthening reactions are carried out and the multiplicity of possible prelengthening reactions are known to the person skilled in the art and are not all explicitly described herein. Reference may be made by way of example to the applications EP 0 508 183 A1 and EP 0 432 087 A1.

A special embodiment relates to those urethane (meth)acrylate resins which are produced in accordance with the method briefly outlined below or as described in the Examples:

The method is a method for the production of vinyl ester urethane resins, especially urethane (meth)acrylate resins (also U(M)A resins hereinbelow), which is characterised in that, as starting material for the production of the vinyl ester urethane resin, especially U(M)A resin, an isocyanate having an average functionality of 2 or less or especially more than 2 (which can also be achieved by mixing isocyanates of a functionality less than two with isocyanates of a functionality greater than 2), for example of 2.0 or especially from 2.1 to 5, for example from 2.2 to 4, advantageously, for example, from 2.3 to 3.5, is reacted with an aliphatic alcohol having at least one C—C double bond (non-conjugated - olefinic bond), especially with a hydroxyalkyl (meth)acrylate, preferably hydroxy-lower alkyl (meth)acrylate, such as hydroxyethyl (meth)acrylate, hydroxybutyl (meth)acrylate or glycerol dimethacrylate or especially hydroxypropyl (meth)acrylate, preferably 2-hydroxypropyl methacrylate (HPMA). The technically available HPMA is to be regarded as a mixture of 2-hydroxypropyl methacrylate and hydroxyisopropyl methacrylate, - other aliphatic alcohols having an olefinic bond can also be present as technical isomeric mixtures or as pure isomers. When an excess of HPMA is used, the excess HPMA can act additionally as reactive diluent.

An isocyanate having an average functionality of less than 2 or 2 or especially more than 2, for example from 2.1 to 5, for example from 2.2 to 4, advantageously, for example, from 2.3 to 3.5, is, for example, a polyisocyanate with uretdione, isocyanurate, iminooxadiazinone, uretonimine, biuret, allophanate and/or carbodiimide structures (advantageously with a molecular weight distribution such that no single molecular species is present in an amount of more than 50 % by weight and at the same time more than 50 % by weight of the chains are composed of at least 3 + 1 covalently bonded monomer units/reactants (see the more precise definition of a polymer according to REACH)) or preferably a mixture (which is typically formed, for example, in technical production processes or is subsequently specifically adjusted (for example by adding and/or distilling off monomers or monomer mixtures)) of (l) one or more monomeric mono- or especially di-isocyanates, such as diphenylmethane diisocyanate (MDI), especially 4,4′-diphenylmethyl diisocyanate or 2,2′-diphenylmethane diisocyanate or mixtures of diphenylmethane diisocyanate isomers (with different positions of the isocyanate groups on the phenyl nuclei), such as those just mentioned, with (ll) one or more “polymeric” diphenylmethane diisocyanates (PMDI), that is to say preferably crude MDI (crude product of the industrial production of MDI without separation of the individual isomers, for example by distillation) with (that is to say including) a plurality of isomers and higher-functional homologues and, for example, an average molecular weight of an order of magnitude of from 200 to 800 g/mol and a functionality as indicated above, for example having an average molecular weight of from 280 to 500, for example from 310 to 480, and a functionality of from 2.4 to 3.4, for example of 3.2. Preference is given to commercially available PMDI that are obtained from the crude MDI itself or are obtained from the crude MDI, for example, by distilling off and/or adding monomeric MDl, and have an average molecular weight of 310-450 and can also include uretdione, isocyanurate, iminooxadiazinone, uretonimine, biuret, allophanate and/or carbodiimide structures. Special preference is given to commercially available PMDI having a molecular weight distribution such that no single molecular species is present in an amount of more than 50% by weight.

“Functionality” is to be understood as being the number of isocyanate groups per molecule; in the case of diphenylmethane diisocyanate this functionality is (substantially, that is to say apart from impurity-related variations) 2; in the case of the PMDI, it is an average functionality (usually indicated by the manufacturer) which can be calculated in accordance with the formula

$$f = \frac{\sum {n}_{i} ⋅ {f}_{i}}{\sum {n}_{i}}$$

(f = functionality, nᵢ = number of molecules of a functionality fᵢ,)

and is preferably between less than 2 or 2 or especially preferably more than 2, for example 2.1, and 5.0 or in the ranges as indicated above.

The method for the production of urethane (meth)acrylate resins is preferably carried out in the presence of a catalyst; appropriate catalysts which catalyse the reaction between hydroxyl groups and isocyanate groups are well known to person skilled in the art, for example a tertiary amine, such as 1,2-dimethylimidazole, diazabicyclooctane, diazabicyclononane, or an organometallic compound (for example of K, Sn, Pb, Bi, Al and especially also of transition metals such as Ti, Zr, Fe, Zn, Cu); and also mixtures of two or more thereof; for example in a proportion (relative to the reaction mixture) of from 0.001 to 2.5 % by weight; preferably in the presence of stabilisers (inhibitors), such as, for example, phenothiazine, TEMPO, TEMPOL, hydroquinone, dimethyl hydroquinone, tert-butyl hydroquinone, hydroquinone monoethyl ether, tert-butyl pyrocatechol and/or p-benzoquinone, and also mixtures of two or more thereof; for example in an amount of from 0.0001 to 2.5 % by weight, relative to the reaction mixture, at preferred temperatures, for example, in the range from 0 to 120° C., advantageously from 50 to 95 (or to 80)°C.

Examples of suitable catalysts and stabilisers are known to the person skilled in the art, for example as can be seen from “Polyurethane Kunststoff-Handbuch 7” [Polyurethanes Plastics Handbook 7] by Becker, G.W.; Braun, D.; Oertel, G., 3rd Edition, Carl Hanser Verlag, 1993.

The reaction can be carried out without solvent (the aliphatic alcohol having at least one C-C double bond, especially the hydroxy-(lower)alkyl (meth)acrylate, then itself serves as solvent) or in the presence of a suitable solvent, for example a further reactive diluent. “Reactive” here relates to the formulation of the adhesive composition and the curing thereof, not to the addition of the alcohol to the isocyanate.

The reaction can also be carried out in such a way that, by means of a prelengthening step, a prepolymer is formed and only afterwards are the isocyanate groups still remaining reacted with the aliphatic alcohol having at least one C—C double bond, especially with the hydroxy-(lower)alkyl (meth)acrylate, as described hereinabove or hereinbelow.

For the production of the prepolymer, to achieve an average isocyanate functionality of two or, especially, greater than two, there are used the above-mentioned isocyanates and polyols having two or more hydroxy groups per molecule and/or polyamines having two or more amino groups per molecule or aminols having two or more amino and hydroxy groups per molecule; or there are used isocyanates having a functionality of 2 with polyols, polyamines or aminols having an average OH and/or amino functionality of more than 2.

Polyols (di- or higher-functional alcohols) here are especially di- or higher-functional alcohols, for example secondary products of ethylene oxide or propylene oxide, such as ethanediol, di-or tri-ethylene glycol, propane-1,2- or -1,3-diol, dipropylene glycol, other diols, such as 1,2-, 1,3- or 1,4-butanediol, 1,6-hexanediol, neopentyl glycol, 2-ethylpropane-1,3-diol or 2,2-bis(4-hydroxycyclohexyl)-propane, triethanolamine, bisphenol A or bisphenol F or the oxyethylation, hydrogenation and/or halogenation products thereof, higher-valent alcohols, such as, for example, glycerol, trimethylolpropane, hexanetriol and pentaerythritol, hydroxyl-group-containing polyethers, for example oligomers of aliphatic or aromatic oxirans and/or higher cyclic ethers, for example ethylene oxide, propylene oxide, styrene oxide and furan, polyethers, in each case having terminal hydroxy, which contain aromatic structural units in the main chain, for example those of bisphenol A or F, hydroxyl-group-containing polyesters based on the above-mentioned alcohols or polyethers and dicarboxylic acids or their anhydrides, for example adipic acid, phthalic acid, isophthalic acid, terephthalic acid, tetra- or hexa-hydrophthalic acid, endomethylenetetrahydrophthalic acid, maleic acid, fumaric acid, itaconic acid, sebacic acid or the like. Special preference is given to hydroxyl compounds having aromatic structural units having a chain-stiffening effect, hydroxy compounds having unsaturated components for increasing the crosslinking density, such as fumaric acid, or branched or star-shaped hydroxy compounds, especially tri- or higher-functional alcohols and/or polyethers or polyesters that contain structural units thereof. Special preference is given to lower alkanediols (yielding divalent -O-lower alkylene-O- radicals).

Aminols (aminoalcohols) are compounds that especially contain one or more hydroxy groups and one or more amino groups in one and the same molecule. Preferred examples are aliphatic aminols, especially hydroxy-lower alkylamines (yielding -NH-lower alkylene-O- or -O-lower alkylene-NH- radicals), such as ethanolamine, diethanolamine or 3-aminopropanol, or aromatic aminols, such as 2-, 3- or 4-aminophenol.

Polyamines (di- or higher-functional amines) are organic amino compounds having 2 or more amino groups, especially hydrazine, N,N′-dimethylhydrazine, aliphatic di- or poly-amines, especially lower alkanediamines (yielding -NH-lower alkyl-NH- radicals), such as ethylenediamine, 1,3-diaminopropane, tetra- or hexa-methylenediamine or diethylenetriamine, or aromatic di- or poly-amines, such as phenylenediamine, 2,4- and 2,6-toluenediamine, or 4,4′- diaminodiphenylmethane, polyether diamines (polyethylene oxides having terminal amino groups) or polyphenyl/polymethylene-polyamines that are obtainable by condensation of anilines with formaldehyde.

The ratio of free isocyanate groups of the isocyanate(s) to hydroxy groups of the hydroxy-lower alkyl (meth)acrylate(s) is advantageously so selected that rapid and complete reaction of the isocyanate groups is obtained, that is to say the molar amount of hydroxy groups (and accordingly the correlating molar amount of hydroxy-lower alkyl (meth)acrylate) is greater than the molar amount of isocyanate groups, for example from 1.03 to 5 times greater, such as, for example, from 1.05 to 4 times greater or from 1.1 to 3 times greater. Excess hydroxy-lower alkyl (meth)acrylate serves as reactive diluent.

The U(M)A resins obtainable by means of the method are those which constitute urethane (meth)acrylate resins present or usable according to the invention as urethane-(meth)acrylate-based reactive resin.

The coating systems and adhesive systems according to the invention, in addition to including the constituents mentioned hitherto, can also include further customary constituents (for example additives or other constituents mentioned hereinabove or hereinbelow). Such further constituents can be present, for example, in an amount of, in total, up to 80 % by weight, preferably between 0.01 and 65 % by weight. Even when “based on” is not explicitly mentioned, such customary constituents are possible. Preferably a urethane methacrylate or, especially, a (meth)acrylate as described above is present as sole free-radical-hardenable synthetic resin (reactive resin).

Examples of further ingredients (additives) of reactive resins in coating systems or adhesive systems having free-radical-polymerisable reactive resins, especially urethane (meth)-acrylates or compounds of the formula (ll), or coating systems and adhesive systems including them, are here metal-salt-based or, preferably, aminic accelerators, inhibitors, non-reactive diluents, reactive diluents, thixotropic agents, fillers and/or further additives, or mixtures of two or more such ingredients.

As aminic accelerators there come into consideration those having sufficiently great activity, such as, especially, (preferably tertiary, especially hydroxyalkylamino-group-substituted) aromatic amines selected from the group selected from epoxyalkylated anilines, toluidines or xylidines, such as, for example, ethoxylated or propoxylated toluidine, aniline or xylidine, for example N,N-bis(hydroxypropyl or hydroxyethyl)-toluidines or dipropoxy-p-toluidine or -xylidines, such as N,N-bis(hydroxypropyl or hydroxyethyl)-p-toluidine, N,N-bis(hydroxyethyl)-xylidine and, very especially, corresponding higher alkoxylated technical products. One or more such accelerators are possible. The accelerators preferably have a content (concentration) of from 0.005 to 10% by weight, especially from 0.1 to 5 % by weight.

As inhibitors there can be added, for example, non-phenolic (anaerobic) and/or phenolic inhibitors.

As phenolic inhibitors (which are often provided as a constituent already mixed in with commercial free-radical-hardening reactive resins but which, furthermore, may also be absent) there come into consideration (non-alkylated or alkylated) hydroquinones, such as hydroquinone, furthermore mono-, di- or tri-methyl hydroquinone, (non-alkylated or alkylated) phenols, such as 4,4′-methylene-bis(2,6-di-tert-butylphenol), 1,3,5-trimethyl-2,4,6-tris(3,5-di-tert-butyl-4-hydroxybenzyl)-benzene, (non-alkylated or alkylated) pyrocatechols such as tert-butyl-pyrocatechol, 3,5-di-tert-butyl-1,2-benzenediol, or mixtures of two or more thereof. These have preferably a content of up to 1 % by weight, especially if present between 0.0001 and 0.5 % by weight, for example between 0.01 and 0.1 % by weight.

As non-phenolic or anaerobic (that is to say, unlike the phenolic inhibitors, active also without oxygen) inhibitors there come into consideration preferably phenothiazine or organic nitroxyl free radicals. As organic nitroxyl free radicals there can be added, for example, those described in DE 199 56 509, which is incorporated herein by reference, especially in respect of the compounds mentioned therein, especially 1-oxyl-2,2,6,6-tetramethylpiperidin-4-ol (“4-OH-TEMPO” or “TEMPOL”). The proportion by weight of the non-phenolic inhibitors is (if present) preferably in the range of from 1 ppm (by weight) to 2 % by weight, especially, for example, in the range of from 5 ppm to 1 % by weight, relative to the reactive resin formulation.

As non-reactive diluents there can be added, for example, vegetable oils, such as castor oil, or furthermore bio-alcohols and fatty acids and esters thereof, or mixtures of two or more thereof, for example in a proportion of from 3 to 60 % by weight, for example from 4 to 55 % by weight.

As thixotropic agents there can be used customary thixotropy-imparting rheology aids, such as pyrogenic silica or silica that has been surface-treated (for example with silanes). They can be added, for example, in a proportion by weight of from 0.01 to 50 % by weight, for example from 0.5 to 20 % by weight.

As fillers there can be present customary fillers having a relatively large average particle size, especially chalks, gypsum, burnt lime, sand, such as quartz sand, quartz powder, corundum, glass (also in the form of hollow beads), porcelain, ceramics, silicates, clays or barite, which can be added in the form of powder, in granular form or in the form of shaped bodies, or others, such as kernel or shell flours obtained from plants, which increases the biogenic carbon content, such as olive stone flour, coconut shell flour or furthermore walnut shell flour, or mixtures of two or more thereof, it being possible for the fillers furthermore or especially also to be silanised. The fillers can be present in one or more components of a multi-component synthetic resin fixing system according to the invention, for example in one or both components of a corresponding two-component kit; the content of fillers is preferably from 0 to 90 % by weight, for example from 10 to 50 % by weight (in the case of the installation of anchoring elements, broken casing material (for example splintered glass or splintered plastics), for example fragments of capsules, can also be counted as filler). In addition or as an alternative to one or more of the mentioned fillers, furthermore, hydraulically hardenable fillers, such as gypsum, burnt lime or cement (for example alumina cement or Portland cement), water glasses or active aluminium hydroxides, or two or more thereof, can be added.

Fillers can be present in one component or, in the case of multi-component compositions, in a plurality of components of a synthetic resin fixing system according to the invention, for example in the form of a multi-component kit (especially a two-component kit). They are present in a proportion of preferably from 0 to 80 % by weight, especially from 5 to 80 % by weight, for example from 40 to 70 % by weight.

Further additives can also be added, such as non-reactive diluting agents, flexibilisers, stabilisers, rheology aids, wetting and dispersing agents, colouring additives, such as dyes or especially pigments, for example for staining the components different colours for better monitoring of their intermixing, or furthermore plasticisers, or mixtures of two or more thereof. Such further additives can preferably be added, in total, in proportions by weight of, in total, from 0 to 90 % by weight, for example from 0 to 40 % by weight.

As “reactive diluents” it is possible for one or more free-radical-hardening unsaturated reactive diluents in biogenic or non-biogenic form to be added, which are to be understood as being primarily those which, as free-radical-curing components (where “curing” includes “curable (for example prior to addition of hardener)”), include organic compounds having unsaturated (for example olefinic) radicals or especially which consist of such compounds, for example especially (meth)acrylate or (meth)acrylamide monomers, such as acrylic acid and/or methacrylic acid or preferably esters thereof (referred to as (meth)acrylates) or amides, especially (meth)acrylates such as mono-, di-, tri- or poly-(meth)acrylates (including hydroxy-lower alkyl (meth)acrylates, which may also already be present as reactive diluents in the event of an excess from the U(M)A resin production according to the invention, such as hydroxypropyl (meth)acrylate or hydroxyethyl (meth)acrylate), alkyl (meth)acrylates having from 1 to 10 (meth)acrylate groups, such as mono-, di-, tri-, tetra-, penta-, hexa- or poly-(meth)-acrylates, for example alkyl di- or tri-(meth)acrylates, such as 1,2-ethanediol di(meth)acrylate, butanediol di(meth)acrylate, such as 1,3- or especially 1,4-butanediol di(meth)acrylate, hexanediol di(meth)acrylate, 1,10-decanediol di(meth)acrylate, diethylglycol di(meth)acrylate, especially preferably oligo-alkylene glycol di(meth)acrylates, as described in DE 10 2014 109355.0, which is incorporated herein by reference, trimethylolpropane tri(meth)acrylate, glycerol tri(meth)acrylate, polyglycerol poly(meth)acrylate, polyethylene glycol di(meth)-acrylate, the reaction product of the acylation of glycerol formal (an equilibrium mixture of 5-hydroxy-1,3-dioxolan and 4-hydroxymethyl-1,3-dioxolan which is liquid at room temperature) with methacrylic acid or the reactive derivatives thereof (known as GLYFOMA from Evonik), cycloalkyl (meth)acrylates, bicycloalkyl (meth)acrylates or heterocyclyl (meth)acrylates, wherein cycloalkyl or bicycloalkyl furthermore can be substituted and has from 5 to 7 ring carbon atoms and heterocyclyl has 5 or 6 ring atoms, furthermore can carry substituents and has 1 or 2 ring hetero atoms selected from N, O and S, such as tetrahydrofurfuryl (meth)acrylate or isobornyl (meth)acrylate, or acetacetoxyalkyl (meth)acrylate; or furthermore styrenes, such as styrene, α-methyl styrene, vinyl toluene, tert-butyl styrene and/or divinyl benzene; or mixtures of two or more thereof, for example (if present) in a proportion by weight of from 0.1 to 90 % by weight, for example between 10 and 80 % by weight, from 30 to 70 % by weight or from 40 to 60 % by weight, in each case relative to the total weight of the mixture of synthetic resin and reactive diluent excluding fillers but, where applicable, including other additives.

The mentioned free-radical-hardenable oligoalkylene glycol di(meth)acrylates are especially those of the formula I,

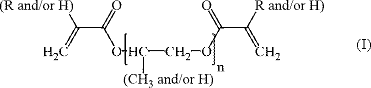

wherein the radicals R independently of one another denote C₁-C₇alkyl, especially methyl, and wherein n denotes, on average, from 2.5 to 13, preferably from 3.5 to 10, especially from 4 to 8 and above all from 4.2 to 7, especially 4.5 and 6.

Examples of corresponding compounds are especially triethylene glycol di(meth)acrylate (TIEGDMA), tetraethylene glycol di(meth)acrylate (TTEGDMA), polyethylene glycol 200-di(meth)acrylate (PEG200DMA) (average value n ≈ 4.5) (most preferred), polyethylene glycol 400-di(meth)acrylate (PEG400DMA) (average value n = 9), furthermore polyethylene glycol 600-di(meth)acrylate (PEG600DMA) (average value n = 13).

In a special and advantageous embodiment of the invention, the hardenable components (comprising reactive resin and the associated hardeners according to the invention (hardener component)) of a coating system or adhesive system according to the invention are stored separately from one another in a two-component or, furthermore, multi-component system or kit before they are mixed with one another for use at the desired location (for example close to or in a hole or crevice, such as a drilled hole).

A multi-component system is understood to be especially a two-component or (furthermore) multi-component kit (preferably a two-component kit) comprising (or in the case of the two-component system consisting of) a component (K1), which includes one or more reactive resins, as described hereinabove and hereinbelow, and the respectively associated hardener as component (K2) as defined above and hereinbelow, it being possible for further additives to be provided in one or both of the components, preferably a two-chamber or, furthermore, multi-chamber device, wherein the components (K1) and (K2) that are able to react with one another and optionally further separate components are present in such a way that their constituents do not come into contact with one another during storage prior to use, but that enables components (K1) and (K2) and optionally further components to be mixed together for fixing at the desired location, for example directly in front of or in a hole, and, if necessary, introduced in such a way that the hardening reaction can take place therein. Also suitable are capsules, such as ampoules arranged one inside the other; and also especially multi-component or especially two-component cartridges (which are likewise especially preferred), the chambers of which contain the plurality of components or, preferably, the two components (especially (K1) and (K2)) of the coating system or adhesive system according to the invention having the compositions mentioned hereinabove and hereinbelow for storage prior to use, the kit in question preferably also including a static mixer. The chambers of the multi-component or, especially, two-component cartridges can be formed purely from plastics or in the form of foil pouches (especially multi-layer foil pouches).

As material for a foil pouch suitable for use according to the invention there can be used plastics, such as compostable plastics, for example based on starch, polyesters, polyester amides, polyurethanes, polyvinyl alcohols, cellulose, lignocellulose, polylactic acid, polyhydroxyalkanoates, or mixtures of two or more such materials. Preference is given to water-proof, moisture-proof and water-vapour-proof materials, especially those which have been metallised (for example by vapour deposition) and/or composite foils made of a plastics foil and a metal foil, especially aluminium foil.

Alternatively (or, in the case of mixed systems (hybrid systems), in addition) to free-radical-hardenable synthetic resins (Aa), the coating systems or adhesive systems according to the invention can comprise as synthetic resin those which contain compounds that are hardenable by polyaddition (Ba), especially those which include (preferably at least, on average, two or more per molecule) epoxy groups (Ba1) or especially isocyanate groups (Ba2).

It is assumed, without being bound to this assumption, that by contact with moisture, for example in aqueous solutions or with atmospheric moisture, imines according to the invention are cleaved, with absorption of water, into the associated amines and ketones or aldehydes, the amino groups then undergoing reaction with the epoxy or isocyanato groups, with polyaddition taking place.

Thus the hardeners and imines according to the invention can also be present or used in the hardening of compounds that are curable by polyaddition, such as, especially, synthetic resins that include isocyanate groups (Ba2) and especially in coating systems or adhesive systems according to the invention.

The epoxy-based reactive synthetic resins (Ba1) that are suitable for use in coating systems or adhesive systems used according to the invention include as epoxy component preferably one based on glycidyl compounds, for example those having an average glycidyl group functionality of 1.5 or more, especially of 2 or more, for example from 2 to 10, which can optionally include further glycidyl ether(s) as reactive diluent. The epoxides of the epoxy component are preferably poly(including di)-glycidyl ethers of at least one polyvalent alcohol or phenol, such as novolak, bisphenol F or bisphenol A, or mixtures of such epoxides, for example obtainable by reaction of the corresponding polyvalent alcohols with epichlorohydrin. Examples are trimethylolpropane triglycidyl ether, novolak epoxy resins, bisphenol A epichlorohydrin resins and/or bisphenol F epichlorohydrin resins, for example having an average molecular weight of ≤ 2000 Da. The epoxy resins can have, for example, an epoxy equivalent of from 120 to 2000, preferably from 150 to 400, such as, especially, from 155 to 195, for example from 165 to 185. The proportion of the total mass of the reactants and additives of the injectable synthetic mortar fixing system is preferably from 5 to below 100 % by weight, especially from 10 to 80 % by weight, from 10 to 70 % by weight or from 10 to 60 % by weight. Also possible are mixtures of two or more of such epoxy components. Suitable epoxy resins, reactive diluents and hardeners are also to be found in the reference work by Lee H and Neville K, “Handbook of Epoxy Resins” (New York: McGraw-Hill), 1982 (those compounds are incorporated herein by reference).

Preferably, reactive synthetic resins that include isocyanate groups (Ba2) suitable for use in the coating systems or adhesive systems used according to the invention are those wherein the isocyanates are defined as above for an “isocyanate having an average functionality of 2 or more than 2”. Special preference is given to those reactive resins which fulfil the REACH condition as defined above and which include a very small content of the monomeric isocyanate groups that are to be avoided as far as possible, which also include prepolymers. Isocyanate prepolymers according to the invention or to be used according to the invention are especially those produced or producible by reaction with polyols with polyfunctional, especially difunctional, isocyanates (“monomers”, which does not exclude a production-related residual content (for example 5 % by weight or less, especially 1 % by weight or less) of oligomers), such as, for example, MDI, especially preferably TDI.

As polyols there are used polyfunctional, especially difunctional, polyols, such as, for example, polyester polyols or, especially preferably, polyalkylene glycol polyols such as polyethylene glycol, polypropylene glycol or polytetramethylene glycol polyols.

The isocyanate prepolymers are preferably producible or, especially, produced by reaction of the polyols with a molar excess of the isocyanates, in a molar ratio of NCO:OH groups of preferably from 1.2 to 3:1, especially preferably close to 2:1, after which excess isocyanate monomers are distilled off to a content of < 1 %, for example 0.5 % and very especially preferably < 0.1 %, for example by high vacuum distillation or thin-layer distillation. Preferably (in further contradistinction to the multi-step reaction in DE 100 55 786 A1) the reaction takes place in one reaction step without further reaction, that is to say the reaction product “isocyanate prepolymer” is not (with prior removal of any residual isocyanate monomers still present) reacted in a second reaction of some of the isocyanate groups still present with diol or polyol to form a further prepolymer having fewer isocyanate groups.

Important examples of possible further ingredients (constituents or additives) for components comprising a reactive resin that includes epoxy or isocyanate groups (Ba) and the hardener component are especially accelerators, inhibitors, stabilisers, reactive diluents, thixotropic agents, fillers and other additives.

As accelerators there come into consideration those having sufficiently high activity, such as heavy metal salts, organic tin, titanium, bismuth and antimony compounds or the like; or (especially in the case of reactive resins that include epoxy groups) aminic accelerators. One or more such accelerators are possible. The accelerators preferably have a content (concentration) of from 0.005 to 10 % by weight, especially from 0.1 to 5% by weight. Special preference is given to DABCO (1,4-diazabicyclo[2.2.2]octane), dibutyltin dilaurate or DBU.

As stabilisers there may be mentioned, for example, HALS stabilisers as described in “Polyurethane, Kunststoff Handbuch” [Polyurethanes, Plastics Handbook], Chapter 3.4.8 Anti-ageing agents, p. 121, 3rd edition, 1993, Editors Gerhard W. Becker and Dietrich Braun, Carl Hanser Verlag Munich Vienna, such as, especially, 1-oxyl-2,2,6,6-tetramethylpiperidin-4-ol (“4-OH-TEMPO”), or sterically hindered phenols (4,4′-di-octyl-diphenylamine) such as cresols (2,6-di-tert-butyl-p-cresol). The proportion by weight of stabilisers is preferably in the range from 0.1 ppm to 2 % by weight, preferably in the range from 1 ppm to 1 % by weight.

As thixotropic agents there can be used customary thixotropy-imparting rheology aids, such as pyrogenic silica (optionally surface-treated to be made hydrophobic) or hydrogenated (solid at room temperature) or modified castor oils. They can be added, for example, in a proportion by weight of from 0.01 to 50 % by weight, for example from 1 to 20 % by weight.

As fillers there are used customary fillers, especially cements (for example Portland cements or high-alumina cements), chalks, sand, quartz sand, aluminium silicates, quartz powder or the like, which can be added in the form of powder, in granular form or in the form of shaped bodies, or others, as mentioned, for example, in WO 02/079341 and WO 02/079293 (which in this respect are incorporated herein by reference), or mixtures thereof, it being possible for the fillers furthermore or, especially, also to be silanised. The fillers can be present in one or more components of an adhesive system according to the invention, for example in one or both components of a corresponding two-component kit. In addition or alternatively, hydraulically hardenable fillers, such as gypsum (for example anhydrite), burnt lime or cement (for example alumina or Portland cement), water glasses or active aluminium hydroxides, or two or more thereof, can be added as filler. The fillers can be present in any desired forms, for example as a powder or flour, or as shaped bodies, for example in the form of cylinders, rings, spheres, hollow spheres, platelets, rods, saddles or crystals, or furthermore in the form of fibres, and the corresponding basic particles preferably have a maximum diameter of from 0.0001 to 10 mm. The proportion of fillers is preferably from 0 to 90 % by weight, for example from 10 to 90 % by weight.

Further additives (additional ingredients) can also be added, such as drying agents (for example zeolite), in order to increase the storage stability of the moisture-sensitive components, plasticisers, such as phthalic acid esters or sebacic acid esters, non-reactive diluents (for example solvents, such as lower alkyl ketones, for example acetone, di-lower alkyl-lower alkanoylamides, such as dimethylacetamide, lower alkyl benzenes, such as xylenes or toluene, or paraffins, or water), flexibilisers, stabilisers, rheology aids, wetting agents, pigments or colourings, or mixtures of two or more of those additives, or the like. Such further additional ingredients can preferably be added, in total, in proportions by weight of, in total, from 0 to 90 % by weight, for example from 0 to 40 % by weight.

According to the invention, in the case of the free-radical polymerisation none of the otherwise customary initiators of free-radical polymerisation, for example free-radical-forming peroxides, for example organic peroxides, such as diacyl peroxides, for example dibenzoyl peroxide, ketone peroxides, such as methyl ethyl ketone peroxide or cyclohexanone peroxide, or alkyl peresters, such as tert-butyl perbenzoate, inorganic peroxides, such as persulfates or perborates, azides, azo compounds or photoinitiators, are admixed, that is to say the subjects of the invention do without such compounds (they are free of such compounds). That does not mean that peroxides will not occasionally be formed in passing during storage or during reaction (for example also as a result of reaction with atmospheric oxygen) but there is no deliberate active addition “from the outside”.

According to the invention the hardener for a free-radical-polymerisable system consists of an initiator system, comprising
(a) at least one activator in the form of a metal salt and as free-radical starter
(b1) an amino-functional polyoxyalkylene (i) as defined in one of the above embodiments in admixture with an aldehyde and/or ketone (ii) as defined in one of the above embodiments; or
(b2) an imine as defined in one of the above embodiments,
or a mixture of constituents (b1) and (b2).

In the case of the compounds that are hardenable by polyaddition, the hardener (b2) includes an imine as defined in one of the above embodiments.

The aldehydes, ketones, amines, aldimines or ketimines used or included according to the invention are known or producible/obtainable by methods known per se or are preferably obtained in accordance therewith, see in this connection also WO 2016/206777 A1.

An amino-functionalised polyoxyalkylene, the polyoxyalkylene chains of which are a copolymer of oxyethylene and oxypropylene units, is preferably either one where the oxyethylene and oxypropylene (which especially denotes 2-methyl-oxyethylene) are randomly distributed in the polyoxyalkylene chain and/or are present as blocks. Special preference is given to compounds having a poly(oxyethylene) moiety which has been lengthened with oligo(propyleneoxy) radicals (especially oligo(2-methylethyleneoxy) at both ends and is “end-capped” at the outer chain ends in each case with a primary amino group.

Special preference is given to compounds of the formula (lll)

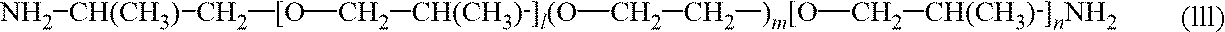

wherein l and n each independently of the other denote, on average, from 1 to 10, preferably approximately 3.6; and m denotes from 1 to 50, preferably from 8 to 10, especially approximately 9, wherein especially,

on average, l and n denote approximately 3.6 and, on average, m denotes approximately 9, with “approximately” preferably denoting a range of variation of ± 0.2, especially of ± 0.1.

The aldehydes and/or ketones are compounds which have at least one or, furthermore, more (primary and/or secondary) hydrogen atoms at the carbon atom in the α-position to the carbonyl group. Aldehydes are preferred to ketones. Examples of such aldehydes are propanal, valeraldehyde, isovaleraldehyde, or methoxyacetaldehyde, or 3,7-dimethyl-6-octenal (citronellal) or 3,7-dimethyl-7-hydroxyoctanal (hydroxycitronellal). As corresponding ketones there may be mentioned here by way of example also methyl isobutyl ketone, acetone, or methyl ethyl ketone or 6-methyl-5-hepten-2-one.

The aldehydes (which are preferred to ketones) and/or ketones are especially preferably compounds which have a double bond and/or branch at the carbon atom in the α-position to the carbonyl group. As a result, the especially preferred aldehydes and/or ketones have only one (tertiary) hydrogen atom at the carbon atom in the α-position to the carbonyl group. Examples of especially preferred aldehydes are isobutyraldehyde (very especially preferred), 2-ethylhexanal, 2-methylbutanal, 2-ethylbutanal, 2-methylvaleraldehyde, 2,3-dimethyl-valeraldehyde, cyclohexylcarboxaldehyde, or 3,7-dimethyl-2,6-octadienal (Citral), 3-(4-tert-butylphenyl)-2-methylpropanal (Lilial, Lysmeral), tetrahydrofuran-3-carboxaldehyde, tetra-hydro-2-furancarboxaldehyde, 4-formyltetrahydropyran, tetrahydro-2H-pyran-2-carbaldehyde or tetrahydropyran-3-carbaldehyde. As especially preferred ketones there may be mentioned here by way of example diisopropyl ketone, 3-methyl-2-pentanone, 2-methylcyclohexanone or β-ionones.

In the case of the activators, constituents used in the form of a metal salt, which also includes metal complexes and metal oxides, are preferably one or more metal salts or, especially, one or more salts of organic and/or inorganic acids with metals, for example selected from copper, iron, vanadium, manganese, cerium, cobalt, zirconium, or bismuth, or mixtures of two or more thereof. In particular, the metal salts are selected from the group consisting of vanadium, iron, manganese and copper, especially in the form of salts or complexes with inorganic acid radicals, such as sulfate and/or carbonate radicals and/or organic acid radicals, for example carboxylate radicals, - the organic acids preferably being saturated - such as carboxylates with CH₃, C₂-C₂₀alkyl, a C₆-C₂₄aryl radical or C₇-C₃₀aralkyl radical, for example octoate, for example 2-ethyl hexanoate (isooctanoate), neodecanoate, or acetylacetonate. Special preference is given to manganese carboxylates, such as Mn acetate or Mn octoate, copper carboxylates, such as copper octoate or copper naphthenate, copper quinolates, iron carboxylates, such as iron octoate and/or vanadium carboxylates and/or the group of metal salts with inorganic acids which comprises, for example, iron chloride, iron sulfate, copper sulfate and copper chloride.

Such activators are known or can be produced according to processes known per se and are preferably present in a proportion of from 0.01 to 20 % by weight, for example from 0.1 to 5 % by weight.

Based on the hardener component, the proportion of the imine in a possible preferred embodiment of the invention in the case of the free-radical polymerisation is from 0.5 to 90 % by weight, especially from 0.9 to 30 % by weight, in the case of the polymerisation by polyaddition from 5 to 95 % by weight, preferably from 10 to 80 % by weight.

The proportion of the hardener in a coating system or adhesive system according to the invention is, in total, preferably in a range of from 1 to 60 % by weight, for example from 2 to 50 % by weight.

Anchoring elements, for use without a drilled hole or crevice (which also includes “recess”), are elements to be fixed onto a surface, such as, for example, adapters etc., or, preferably for use in the chemical fixing of anchoring elements in holes (especially drilled holes) or recesses (such as crevices), are those made of metal, such as undercut anchors, threaded rods, screws, drill anchors, bolts or the like; furthermore, they can also be nails or other pin-like connecting elements.

Parameters, insofar as they are mentioned in the context of the present application, are determined in accordance with methods known to the person skilled in the art, especially as described in the Examples.

### Experimental Part

The following Examples illustrate the invention but do not limit the scope thereof; however, they do themselves constitute advantageous embodiments of the invention.

In the following Examples, those constituents of the model formulations which are not labelled are mixed in advance. The polymerisation is initiated by addition and mixing-in of the constituent labelled with “^{x}”.

The methods for determining parameters also apply to the general part of the description:

### Determination of the Gel Time

The determination of the gel time is carried out manually with a commercially available stopwatch and a commercially available thermometer at room temperature (about 23° C.). For that purpose all the constituents are mixed and the temperature of the sample is measured immediately after mixing. The sample itself is located in a plastics beaker. Evaluation is carried out in accordance with DlN 16945 (1989-03). The gel time is the time at which the temperature rises above 35° C. This corresponds to a rise in temperaure of about 10 K.

### Pull-out Tests From Concrete

For pull-out tests using threaded rods M12, the following procedure, in accordance with ETAG 001 PART 5 (Publisher DIBT, Berlin 2008), is carried out:

First of all, a hammer drill is used to drill holes (diameter 14 mm; depth 84 mm) in a concrete test specimen (concrete type C20/25) lying in a horizontal position. The drilled holes are cleaned using a hand blower and a hand brush. The drilled holes are then filled two-thirds full with the particular hardenable composition for fixing purposes to be tested. A threaded rod is pushed into each drilled hole by hand. The excess mortar is removed using a trowel. After 1 hour at room temperature, the threaded rod is subjected to pulling until failure occurs, the failure load being measured.

### Determination of the Tensile Shear Strength

The final strength after 24 hours is determined with the aid of the tensile shear strength according to DIN EN ISO 4587 (2003-03). For that purpose, beechwood test specimens (100 × 25 × 5 mm) are adhesively bonded to one another (adhesively bonded surface area 13 × 25 mm) and after 24 h curing time at a temperature of 23° C. tested with a universal testing machine Z010 by Zwick GmbH & Co. KG (BT1-FR010TH.A50).

During use or during the procedures, the mixture consisting of the adhesive system can be applied to the surfaces to be adhesively bonded over the whole of their common surface area or only in some regions thereof.

For the production of the following exemplary formulations, the following constituents and abbreviations are used.

TABLE 1

Constituents and abbreviations used

| | |
|---|---|
| Abbreviation | Item |
| HRI | Resin formulation l |
| BDDMA | 1,4-Butanediol dimethacrylate |
| HPMA | 2-Hydroxypropyl methacrylate |
| VPLS2142 | CSTICO®phen VP LS 2142 (csc jäklechemie GmbH & Co. KG) (isophoronediamine-isobutyraldimine or 1,3,3-trimethyl-N-(2-methylpropylidene)-5-[(2-methylpropylidene)amino]cyclohexanemethylamine) |
| VPLS2965A | Desmophen VPLS2965A (BayerMaterialScience) (ketimine from isophoronediamine and methyl isobutyl ketone) |
| Isob | Isobutyraldehyde |
| ED600 | Jeffamine ED600 (Huntsman Corporation, The Woodlands, Texas, USA) (aliphatic polyether diamine, obtained from a propylene-oxide-capped polyethylene glycol) formula: NH₂—CH(CH₃)—CH₂—[O—CH₂—CH(CH₃)—]—(O—CH₂— CH₂—)ₘ[O—CH₂—CH(CH₃)—]ₙNH₂ |
| EDR148 | Jeffamine EDR-148 (Huntsman Corporation, The Woodlands, Texas, USA) (symmetrical, unhindered diamine with polyethylene glycol with 2-aminoethoxy terminal groups, MW 148) |
| | 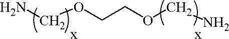 |
| D230 | Jeffamine D230 (Huntsman Corporation, The Woodlands, Texas, USA) (difunctional primary polyether amine with oxypropylene units in the polyether backbone) |
| | 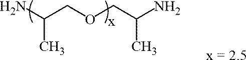 |
| SolCop8 | Soligen Copper 8 (Cu carboxylate, active content: 7.8 - 8.2 %, OMG Borchers GmbH) |
| Mn(lll) | Manganese(lll) acetate 3% in HPMA |
| OxyC | Borchers Oxy Coat (highly activated iron; OMG Borchers GmbH) |
| CF31 | Nouryact CF31 (metal complex, Akzo Nobel Functional Chemicals B.V.) |
| Iro7/8 | Octa Soligen Iron ⅞ (iron carboxylate, active content: 7.3 - 7.7%, OMG Borchers GmbH) |
| SolMn6 | Soligen Manganese 6 (Manganese naphthenate, active content: 6 %, OMG Borchers GmbH) |
| END15 | Umicore Ecos ND15 (cobalt-containing polymer dry substance, active content: 4 %, Umicore Specialty Materials Brugge NV) |
| CuSO4 H2O | Copper sulphate pentahydrate 8% in water |
| CuSO4 MEG | Copper sulphate pentahydrate 8 % in monoethylene glycol |
| BDC8 | Borchers Deca Copper 8 (Cu neodecanoate, active content: 7.8 - 8.2%, OMG Borchers GmbH) |
| LM20R | Lupranat M20 R (product based on 4,4′-diphenylmethane diisocyanate (MDI) with higher-functional oligomers and isomers, average functionality 2.7; BASF) |

### Example 1: General Working Procedure L: Synthesis of Aldimines

The amine in question is introduced as initial charge into a round-bottomed flask. With vigorous stirring, the aldehyde is slowly added from a dropping funnel, during which the temperature of the mixture increases. For use of the aldimine according to the invention as initiator, the volatile constituents need not be removed. Depending upon the amine used (“hydrophobic” amines), the water of reaction formed separates from the aldimine. The residual water remaining in the aldimine does not interfere with its use as initiator. When “hydrophilic” amines are used, no phase separation takes place. For use as hardener for polyaddition-hardenable reactive resins, the volatile constituents are removed by distillation under reduced pressure. Following distillation, the aldimine is additionally dried over molecular sieve. If desired, it is also possible for the aldehyde to be introduced as initial charge and the amine added dropwise. The reaction (freedom from carbonyl and imine groups detectable by lR spectroscopy) is checked by means of FT-IR.

### Example 2: Simplified Resin Formulation

The following starting materials are mixed together for the simplified resin formulations:

TABLE 2

Simplified resin formulation l (HRI)

| Starting material | % by weight |
|---|---|
| Ethoxylated bisphenol-A-dimethacrylate* | 88.76 |
| BDDMA | 7.14 |
| HPMA | 4.10 |

*technical product - degrees of ethoxylation may vary very slightly (but are always the same in the Examples given)

### Example 3: Determination of the Gel Times of the Aldimine According to the Invention In comparison with VPLS2142 with free-radical starter b2)

In order to illustrate the advantages of the aldimine according to the invention as hardener system imine-metal salt for cold-hardening vinyl ester resins in comparison with commercially available aldimines and ketimines, exemplary formulations are prepared with the resin formulation l described above and various metal salts. As free-radical starter the aldimine according to the invention obtained from isobutyraldehyde in admixture with Jeffamine ED600 is used. The commecially available aldimine (CSTICO®phen VP LS 2142) consists of isobutyraldehyde and isophorondiamine and the ketimine provided as exemplar (Desmophen VPLS2965A) consists of methyl isobutyl ketone and isophoronediamine. Table 3 belows lists the gel times and the maximum temperatures achieved during the polymerisations:

TABLE 3

Gel times determined with free-radical starters b2) and different metal salts

| Test name | HRI [g] | b2) VPLS21 42/2965 A [g] | b2) Isob-ED600 | Metal salt [g] | Metal | t Temp. > 35° C. [hh:mm:ss] | Tmax [°C] |
|---|---|---|---|---|---|---|---|
| VPLS2142-004 | 9.75 | 0.25^{x} | - | 0.10 | SolCop8 | 00:00:57 | 89.1 |
| ED600-004 | 9.75 | - | 0.64^{x} | 0.10 | SolCop8 | 00:00:50 | 92.5 |
| VPLS2965A-001 | 9.75 | 0.25^{x} | | 0.10 | SolCop8 | not hard after 12h | - |
| VPLS2142-006 | 9.75 | 0.25^{x} | - | 0.10 | Mn(lll) | 00:02:06 | 74.9 |
| ED600-006 | 9.75 | | 064^{x} | 0.10 | Mn(lll) | 00:01:36 | 79.4 |
| VPLS2142-007 | 9.75 | 0.25^{x} | - | 0.10 | OxyC | 00:02:17 | 71.0 |
| ED600-007 | 9.75 | - | 0.64^{x} | 0.10 | OxyC | 00:00:20 | 103.1 |
| VPLS2142-010 | 9.75 | 0.25^{x} | - | 0.10 | CF31 | 00:01:23 | 88.7 |
| ED600-010 | 9.75 | - | 0.64^{x} | 0.10 | CF31 | 00:01:13 | 91.9 |

^{x} added as hardening initiator

It will be seen by reference to Table 3 that the use of equimolar amounts of imine with the aldimine according to the invention achieves significantly more exothermic and more rapid polymerisations of vinyl ester resins at room temperature. Without wishing to be bound to this theory, it is assumed that this can be attributed to the chemical structure of the aldimine or amine used according to the invention, which is a copolymer of oxyethylene and oxypropylene units (aminooxypropylene-capped poly(oxyethylene). The ketimine obtained from isophoronediamine and methyl isobutyl ketone resulted in no polymerisation within 12 hours.

### Example 4: Gel Times of the Amine According to the Invention With Free-radical Starters B1) And different metal salts

In order further to demonstrate the advantages of the aldimine or amine according to the invention shown in Example 3, gel times are determined using free-radical starters b1) in combination with different metal salts. As comparison amine, a pure polyoxypropylenediamine (D230) is used in order to reinforce the above-mentioned theory. Exemplary formulations are produced with resin formulation l and various metal salts. Table 4 below shows the gel times and the maximum temperatures achieved during the curing reactions.

TABLE 4

Gel times determined with free-radical starter constituents (mixture of amine and Isob) (with in situ synthesis of the imines) and different metal salts

| Test name | HRI [g] | b1) D230 [g] | b1) ED600 [g] | Metal [g] | Isob [g] | Metal | t Temp. > 35° C. [hh:mm:ss] | Tmax [°C] |
|---|---|---|---|---|---|---|---|---|
| Process-Metals-001 | 9.75 | 0.24 | - | 0.10 | 0.15^{x} | SolCop 8 | 00:02:35 | 99.6 |
| Process-Metals-ED600-17 | 9.75 | - | 0.63 | 0.10 | 0.15^{x} | SolCop 8 | 00:02:28 | 100.5 |
| Process-Metals-002 | 9.75 | 0.24 | - | 0.10 | 0.15^{x} | Iro7/8 | 00:07:16 | 69.6 |
| Process-Metals-ED600-18 | 9.75 | - | 0.63 | 0.10 | 0.15^{x} | Iro7/8 | 00:04:23 | 70.0 |
| Process-Metals-003 | 9.75 | 0.24 | - | 0.10 | 0.15^{x} | OxyC | 00:02:56 | 71.1 |
| Process-Metals-ED600-19 | 9.75 | - | 0.63 | 0.10 | 0.15^{x} | OxyC | 00:01:51 | 80.0 |
| Process-Metals-005 | 9.75 | 0.24 | - | 0.10 | 0.15^{x} | SolMn6 | 00:03:30 | 92.3 |
| Process-Metals-ED600-21 | 9.75 | - | 0.63 | 0.10 | 0.15^{x} | SolMn6 | 00:00:47 | 95.9 |
| Process-Metals-011 | 9.75 | 0.24 | - | 0.10 | 0.15^{x} | END15 | 00:13:16 | 83.2 |
| Process-Metals-ED600-22 | 9.75 | - | 0.63 | 0.10 | 0.15^{x} | END15 | 00:02:11 | 94.3 |
| Process-Metals-015 | 9.75 | 0.24 | - | 0.10 | 0.15^{x} | CuSO4 H2O | 00:05:14 | 84.6 |
| Process-Metals-ED600-23 | 9.75 | - | 0.63 | 0.10 | 0.15^{x} | CuSO4 H2O | 00:03:18 | 92.1 |
| Process-Metals-016 | 9.75 | 0.24 | - | 0.10 | 0.15^{x} | CuSO4 MEG | 00:05:06 | 92.8 |
| Process-Metals-ED600-24 | 9.75 | - | 0.63 | 0.10 | 0.15^{x} | CuSO4 MEG | 00:02:51 | 93.1 |

^{x} added as hardening initiator

Table 4 shows that the aldimine or amine according to the invention, which is a copolymer of oxyethylene and oxypropylene units, initiates significantly more exothermic and more rapid polymerisations. Here too, equimolar amounts of imine are used. In addition, Table 4 shows that polymerisations of unsaturated reactive resins are initiated at room temperature also with free-radical starters b1) in combination with metal salts.

### Example 5: Pull-out Tests From Concrete With Free-radical Starters B2)

Setting tests are carried out in accordance with the afore-mentioned parameters for “pull-out tests from concrete”.

TABLE 5

Formulations in % by weight for carrying out setting tests and bond stresses determined with free-radical starters b2

| Item | ED600-lsob | D230-lsob | EDR148-lsob |
|---|---|---|---|
| HRI | 32.13 | 34.02 | 34.59 |
| Additive | 0.24 | 0.24 | 0.24 |
| Aldimine | 3.87 | 1.97 | 1.40 |
| BDC8 | 0.56^{x} | 0.56^{x} | 0.56^{x} |
| Fillers | 61.35 | 61.35 | 61.35 |
| Thixotropic agent | 1.86 | 1.86 | 1.86 |
| n (C═N) / 100 g | 0.011 | 0.011 | 0.011 |
| Bond stress [N/mm²] | 20.9 | 17.1 | 18.1 |

^{x} added as hardening initiator

The starting materials of the aldimines can be respectively assigned by reference to the abbreviations used in the items column and Table 1 above for abbreviations. Table 5 demonstrates that the copolymer structure of the aldimine or amine according to the invention composed of oxyethylene and oxypropylene units results in higher bond stresses in direct comparison with pure oxypropylene-(D230) and oxyethylene-(EDR148) aldimines or amines.

### Example 6: Tensile Shear Strengths of the Aldimine (b2) According to the Invention With polyaddition-hardenable reactive resins (Ba2)

In order to demonstrate the applicability of the aldimine according to the invention as adhesive system, beech test specimens are adhesively bonded to one another and the tensile shear strength is determined after 24 h. Table 6 below shows the compositions and the tensile shear strengths determined.

TABLE 6

Determined tensile shear strengths of the aldimine according to the invention (b2)

| Item | VKS20-06 | Mix-01 |
|---|---|---|
| LM20R [% by weight] | 24.5 | 19.4 |
| Isocyanate prepolymer ¹ [% by weight] | - | 9.7 |
| Aldimine (Isob-ED600) [% by weight] | 48.5 | 44.0 |
| Aluminium silicate [% by weight] | 25.0 | 25.0 |
| Pyrogenic surface-treated silica [% by weight] | 2.0 | 1.8 |
| Tensile shear strength [MPa] | 4.1 | 1.5 |

¹ Isocyanate prepolymer: TDI-based, NCO content: 11.1 %, viscosity at 23° C., spindle 7, 10 rev/min in accordance with DIN EN ISO 2555: 94,400 mPa·s, residual monomer content < 0.1 %.

1. An imine, obtainable by reaction of (i) an amino-functionalised polyoxyalkylene, the polyoxyalkylene chains of which are a copolymer of oxyethylene and oxypropylene units, those polyoxyalkylene chains carrying alkoxy groups having primary amino groups, and (ii) a ketone and/or aldehyde having a hydrogen atom at the carbon atom in the alpha-position to the carbonyl group.

2. The imine according to claim 1, which is obtainable by reaction of the amino-functionalised polyoxyalkylene (i), which carries a primary amino group in each case bonded to the terminal oxyalkylene group, with an alkanaldehyde (ii) having a hydrogen atom at the carbon atom in the alpha-position to the carbonyl group.

3. The imine according to claim 1, obtainable by reaction of the amino-functionalised polyoxyalkylene (i) with isobutyraldehyde (ii).

4. The imine according to claim 1, wherein the amino-functionalised polyoxyalkylene (i) carries two 2-aminopropyloxy groups which are terminal in each case.

5. The imine according to claim 1, wherein the amino-functionalised polyoxyalkylene (i) has the following formula: 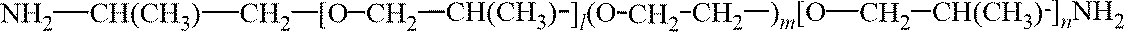
wherein I and n each independently of the other denote, on average, from 1 to 10,; and m denotes from 1 to 50.

6. The imine according to claim 5, in wherein in the formula, on average, l and n denote approximately 3.6 and, on average, m denotes approximately 9.

7. The imine according to claim 1, wherein it has an average molecular weight of 2000 g/mol or less.

8. The imine according to claim 1 in admixture with its starting materials (i) amino-functional polyoxyalkylene and (ii) ketone and/or, aldehyde.

9. A mixture of (i) an amino-functional polyoxyalkylene and (ii) an aldehyde (preferred) and/or ketone, in each case as defined in claim 1.

10. A mixture according to claim 9 as constituent of a coating system or adhesive system or adhesive.

11. A hardener as initiator for a free-radical-hardening system and/or for compounds that are hardenable by polyaddition, which
in the case of the free-radical-hardenable compounds (Case A) includes the following initiator system:
(a) at least one activator in the form of a metal salt and as free-radical starter
(b1) an amino-functional polyoxyalkylene (i) as defined in one of the above embodiments in admixture with an aldehyde (preferred) and/or ketone (ii) as defined in one of the above embodiments; or
(b2) an imine as defined in claim 1,
or a mixture of constituents (b1) and (b2); or
in the case of the compounds that are hardenable by polyaddition (Case B) represents the following constituent:
(b2) an imine as defined in claim 1.

12. A coating system or, adhesive system, including a hardener according to claim 11 and, as reactive resin, either (Aa) a free-radical-polymerisable reactive resin (Case A) or a reactive resin that includes epoxy groups (Ba1) or isocyanate groups (Ba2) (Case B), or a mixture of (Aa) and (Ba).

13. A coating system or, adhesive system according to claim 12, wherein the reactive resin is a free-radical-hardenable reactive resin according to (Aa), of the formula 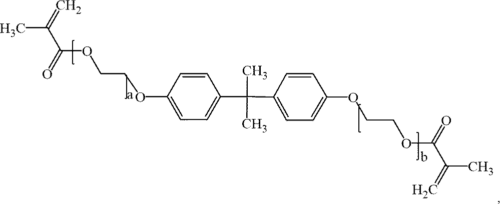
in which a and b each independently of the other denote a number greater than or equal to 0, with the proviso that at least one of the values is greater than 0.

14. A coating system or adhesive system according to claim 12, Case B, wherein the reactive resin is a reactive resin that includes isocyanate groups in accordance with (Ba2), and particularly a prepolymer that includes at least two isocyanate groups.

15. A coating system or adhesive system according to claim 12 in the form of a multi-component system, which includes a reactive resin according to (Aa) or (Ba) in one component and the hardener according to claim 12 in another component.

16. A coatiing system or, adhesive system according to claim 14 in the form of a single-component system, and particularly in the form of a surface-coating that cures by polyaddition or in the form of an adhesive that cures by polyaddition.

17. A method of usinng an adhesive system according to claim 12
(a) for adhesively bonding articles to one another or
(b) for adhesively bonding an article to a fixed substrate.

18. A method of using an adhesive system according to claim 17, Case (b), wherein
an article is to be understood as being an accessory for a swimming pool, bedroom, bathroom or kitchen or an adapter element for holding such an accessory; a plate or a block, preferably made of metal, glass, plastics, concrete or stone; fibres, especially high-modulus fibres, preferably carbon fibres, especially for reinforcing built structures, for example walls, pillars or ceilings or floors; a structural element, such as a panel, a disc, or a block, for example made of stone, glass or plastics; or - in each case implemented in planar form as a mat and as loose material or as composite fabric or material - a non-woven, a woven fabric, a knitted fabric, a planar material obtained by knitting, a material obtained by tufting, preferably in each case with high-modulus fibres, for example made of glass fibres, carbon fibres and/or synthetic fibres, in the case of a composite material having a synthetic matrix; and
a substrate is to be understood as being a building substrate, especially a wall, a ceiling or a floor in the construction field, preferably made of masonry, concrete, unglazed or glazed ceramics, or furthermore made of glass, wood, metal or plastics.

19. A method of using an adhesive system according to claim 18 for adhesively bonding an anchoring element in a hole or a recess in the substrate.

20. A method of using a coating system according to claim 12 for coating a substrate, the substrate.

21. A method for coating a substrate with a coating system according to claim 12, wherein the coating system is applied, if necessary with or after mixing of components thereof that do not cure on their own, and cured, with polymerisation taking place.

22. A method
(a) for adhesively bonding articles to one another or
(b) for adhesively bonding an article to a fixed substrate,
wherein an adhesive system according to claim 12 is applied, if necessary with or after mixing of components thereof that otherwise do not react with one another, to one or more areas to be adhesively bonded of one or more of the articles to be adhesively bonded, and areas to be adhesively bonded are brought into contact with one another and the adhesive system is cured, with polymerisation taking place.
